(19) United States
(12) Patent Application Publication
(10) Pub. No.: US 2006/0155361 A1
(43) Pub. Date: Jul. 13, 2006

(54) Stent with rough surface and its manufacture

(75) Inventors: Albert Schomig, Munchen (DE); Adnan Kastrati, Munchen (DE); Randolf Von Oepen, Los Altos Hills, CA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: ABBOTT LABORATORIES VASCULAR ENTERPRISES LIMITED, Dublin 2 (IE)

(21) Appl. No.: 10/528,352

(22) PCT Filed: Sep. 22, 2003
(86) PCT No.: PCT/EP03/10542

§ 371 Date: Feb. 27, 2006

(30) Foreign Application Priority Data
Sep. 20, 2002 (EP) 02021067.0

(51) Int. Cl. A61F 2/82; B05D 3/12

(52) U.S. Cl. 623/1.15; 623/1.42; 427/2.25

(57) ABSTRACT

A stent is provided wherein at least an outer surface portion is roughened to a predetermined extent and wherein a drug or a therapeutic agent can be applied to said surface. This results in an improved stent, which can be manufactured at low costs and which can further avoid thrombus formation and a stenosis.

The present invention relates generally to stents which are implantable or deployable in a vascular or endoluminal location within the body of a patient to maintain the lumen open and unoccluded at that location, and in particular to improvements in stents.

First of all, stents are widely used for numerous applications where the stent is placed in the lumen of a patient and expanded. Said stents may be used in coronary or other vasculature as well as within the urinary tract, the bial tract and the intestinal tract among other body passageways and conduits.

Commonly, stents are cylindrical members which are expanded from reduced diameters to enlarged diameters. Frequently, such stents are placed on a balloon catheter with the stent in a reduced diameter state. To prevent that the balloon is damaged because of sharp corners and burrs on the surface of the stent and further to avoid thrombus formation, stents are highly polished. This is done for example by sandblasting the surface to remove said imperfections and polishing the stent afterwards to get a smooth surface. Unfortunately, the balloon catheder mounted stent can be dislodged from the uninflated balloon as a result of the navigation through the vessel of the body to the preselected site for deployment because of the highly polished surface. Furthermore, the polished and smooth surface used to avoid thrombus formation has the disadvantage, that endothelial cells have difficulties to ingrow the stent which can result in that restenosis occurs.

In the prior art, some stents are sandblasted on their interior surface to improve stent retention on the balloon.

In U.S. Pat. No. 6,254,631 B1 a stent is disclosed, wherein the exterior surface of the stent is polished such that a smooth surface finish is achieved. The interior surface having a rough surface finish is rougher than the surface finish of the exterior surface to enhance the friction between the stent and the balloon.

Further in U.S. Pat. No. 6,217,607 B1 a stent is disclosed which is provided with a first layer of nobel metal. Further a second outermost layer is provided which is composed of a ceramic like metal such as iridium oxid or titanium nitrate. The second layer is formed with a rough surface to provide an increased friction factor and retention on a balloon during advancement of the stent delivery system through the vessel.

In WO 99/07308 a stent is disclosed wherein a portion of a stent supporting structure is encapsulated with a thin flexible coating made of a polymer which can be used as a carrier for supporting therapeutic agents and drugs. Furthermore, the supporting structure, preferably only the portion which is not encapsulated by the thin flexible coating, is further processed to form a porous exterior surface. Said porous exterior surface renders the exposed portions of the supporting structure, such as the proximal and distal ends more biocompatible by promoting tissue in-growth while reducing the formation of blood clots.

Said stents of the prior art have the disadvantage that they are complicated to manufacture and expensive.

The object of the present invention is therefore to provide an improved stent, which can be manufactured at low costs and which can further avoid thrombus formation and a stenosis.

This object is achieved by the features of the claims.

According to the invention a stent is provided comprising at least an outer surface portion which is roughened to a predetermined extent and wherein a drug or a therapeutic agent can be applied to said surface.

This has the advantage, that the stent does not have to be provided with an additional drug deposit e.g. a polymer layer suitable to carry a drug or therapeutic agent. Instead the drug can be applied directly to the rough surface and released over a predetermined time after the stent has been placed in a desired location of a lumen. Furthermore a roughened exterior surface decrease in-stent restenosis, since cells can attach more easily to said surface than to a smooth one which results in that endothelial ingrowth is accelerated. Intima cells can grow on the rough surface and attach themselves, wherein the endothelialization of the vessel or lumen is promoted. The endothelial cell layer is very smooth and therefore thrombus formation and a stenosis can be avoided.

In a preferred embodiment the drug e.g. Tacrolimus is applied to the rough surface by spraying. This has the advantage, that the application of the drug is effective, simple and inexpensive.

In a further preferred embodiment of the invention imperfections such as e.g. burrs are removed before at least a portion of the surface of the stent is roughened. This has the advantage that the surface can be roughened more uniform which leads to better flow dynamics. Thus less turbulences can occur on the surface which results in a reduction of restenosis.

In a preferred embodiment of the invention the surface is roughened to a predetermined extent by sandblasting. Moreover, sandblasting results in an improvement of the fatigue behaviour. Further the durability of the stent and the surface bonding can be improved. The rough surface also provides an increased surface area for an attachment of a drug or therapeutic agent. Further a stent with a thinner wall with higher radial force and therefore less material can be achieved which also leads to a decrease of restenosis. With sandblasting the surface can be better controlled and produced and further a more uniform and trauma less surface can be achieved.

Furthermore, the use of corundum for sandblasting results in a surface which is technically different from a normal sandblasted surface. It has the advantage that less energy has to be used and/or less time for this finishing sandblasting than for a sandblasting process to remove burrs. Further the sandblasted surface has less depth with regard to the “cavities”. Furthermore the chemical behaviour of such a stent is different from commonly known electropolished stents. The surface chemistry is different due to the incorporation of sand particles into the surface. An immediately repassivated surface leads to more chemically stable passive layers than surfaces which have been passivated in equilibrium.

When blasting the surface the resulting lattice imperfections (e.g. vacancy, dislocation) and further possible phase transitions lead to an increased surface energy and thus to a surface which is chemically more reactive. This can lead to a faster chemical running and/or to additional chemical reactions than in the equilibrium.

In a further preferred embodiment the stent is annealed after the surface has been roughened to a predetermined extent to make him more flexible.

The invention will now be described with reference to the figures, in which

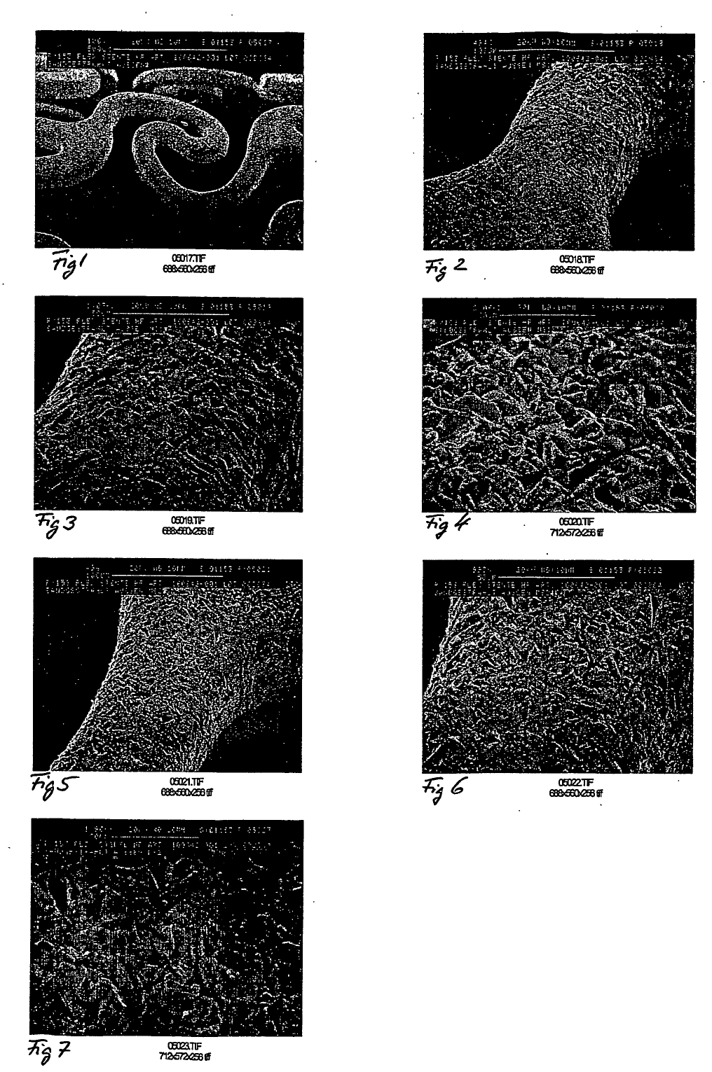

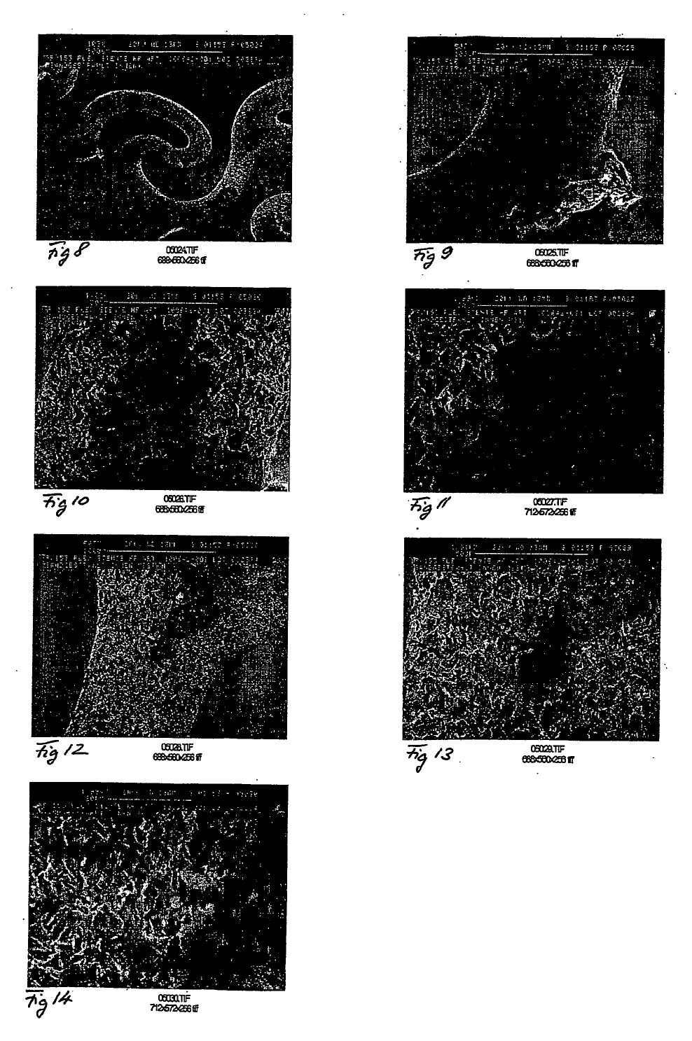

FIGS. 1 to 7 show sandblasted exterior and side surfaces of a stent in different resolutions,

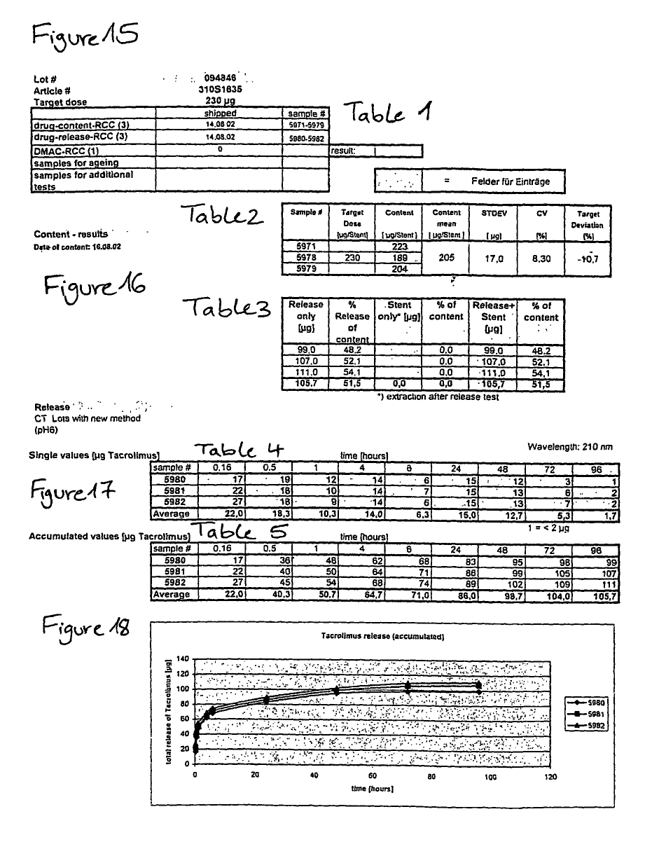

FIGS. 8 to 14 show sandblasted interior and side surfaces of a stent in different resolutions,

FIG. 15 shows a table 1, including a list of samples of stents which are used for studying content and release of a drug applied on their surface,

FIG. 16 show tables 2 and 3 wherein the results of several samples regarding their content are listed,

FIG. 17 show tables 4 and 5, wherein samples are studied regarding release of the drug Tacrolimus, and

FIG. 18 shows a diagram, wherein the release of Tacrolimus of samples with respect to time is shown.

In an embodiment of a stent according to the invention, as shown in FIGS. 1 to 14, the complete surface, i.e. exterior surface, interior surface and side surfaces, is sandblasted by using corundum. It is obvious that the invention is not limited to said embodiment and that also only portions of the surface can be roughened.

In FIGS. 15 to 18 content and release of samples of stents are studied. In this connection normal manufactured stents are compared with stents which are further processed according to the invention. Based on the examples shown in the figures the improved properties of stents according to the invention can be demonstrated.

1. A stent system for placement in a body lumen which is expandable from a contracted state to an expanded state, comprising:
a stent having an exterior surface, an interior surface and side surfaces,
wherein at least a portion of the exterior surface is roughened to a predetermined extent for coating with a drug.

2. The stent system according to claim 1, wherein at least a portion of the interior surface is roughened to a predetermined extent.

3. The stent system according to claim 1, wherein at least a portion of the side surfaces is roughened to a predetermined extent.

4. The stent system according to claim 1, wherein the exterior surface, the interior surface, or the side surfaces are coated with Tacrolimus.

5. The stent system according to claim 1, wherein the said stent comprises a stainless steel.

6. The stent system according to claim 1 further comprising a balloon catheter for delivering the stent.

7. A method for fabricating a stent for placement in a body lumen, the method comprising the following steps:
a) forming a tube which can be deployed from a contracted state to an expanded state, the stent having an exterior surface, an interior surface and side surfaces;
b) roughening of at least a portion of the exterior surface to a predetermined extent; and
c) coating of said surface with a drug.

8. The method according to claim 7 further comprising forming the tube so that at least a portion of the interior surface is roughened to a predetermined extent.

9. The method according to claim 7 further comprising forming the tube so that at least a portion of the side surfaces is roughened to a predetermined extent.

10. The method according to claim 7 further comprising roughening the surface to a predetermined extent by sandblasting.

11. The method according to claim 7, further comprising removing imperfections from the surface of the tube.

12. The method according to claim 11, wherein the step of removing imperfections comprises removing the imperfections by burring.

13. The method according to claim 11, wherein the step of removing imperfections comprises removing the imperfections by electropolishing.

14. The method according to claim 11, wherein the step of removing imperfections comprises removing the imperfections by sandblasting.

15. The method according to claim 14, wherein the step of sandblasting uses sand.

16. The method according to claim 14, wherein the step of sandblasting uses glass beads.

17. The method according to claim 14, wherein the step of sandblasting uses corundum.

18. The method according to claim 7, wherein coating said surface comprises coating said surface with Tacrolimus.

19. The method according to claim 7, wherein coating said surface comprises spraying the drug on said surface.

20. The method according to claim 7, wherein forming the tube comprises forming the tube from annealed material.
